(19) United States
(12) Patent Application Publication
(10) Pub. No.: US 2016/0242947 A1
(43) Pub. Date: Aug. 25, 2016

(54) SPLIT FOOTPLATE, ANKLE BRACES AND BRACING SYSTEMS INCLUDING THE SAME AND ASSOCIATED METHODS

(71) Applicant: Cropper Medical, Inc., Ashland, OR (US)

(72) Inventor: Joseph T. Zachariasen, Talent, OR (US)

(21) Appl. No.: 15/050,151

(22) Filed: Feb. 22, 2016

Related U.S. Application Data
(60) Provisional application No. 62/119,098, filed on Feb. 20, 2015.

(51) Int. Cl. A61F 5/01

(52) U.S. Cl. CPC A61F 5/0127; A61F 2005/0137

(57) ABSTRACT

An ankle brace includes a footplate with a lateral element, a medial element and an expandable element. The lateral element and the medial element of the footplate, which may be formed from a somewhat rigid material, are separate from one another and are spaced apart from one another. The expandable element is located between the lateral element and the medial element. It secures the lateral element and the medial element of the split footplate to one another. Braces and bracing systems that include the split footplate are disclosed, as are methods for supporting an individual's foot and/or ankle.

### CROSS-REFERENCE TO RELATED APPLICATION

A claim for priority to the Feb. 20, 2015, filing date of U.S. Provisional Patent Application 62/119,098, titled SPLIT FOOTPLATE, ANKLE BRACES AND BRACING SYSTEMS INCLUDING THE SAME AND ASSOCIATED METHODS (“the '098 Provisional Application”), is hereby made pursuant to 35 U.S.C. §119(e). The entire disclosure of the '098 Provisional Application is hereby incorporated herein.

### TECHNICAL FIELD

This disclosure relates generally to footplates for ankle braces and, more specifically, to footplates that are configured to be mass produced while providing for a custom fit. Even more specifically, this disclosure relates to footplates with separate lateral and medial elements and an expandable element between the lateral and medial elements. In addition, this disclosure relates to ankle braces and other types of bracing systems that include such a footplate, as well as to methods in which the disclosed footplate is used.

### SUMMARY

An ankle brace according to this disclosure includes a footplate with a lateral element, a medial element and an expandable element. The lateral element and the medial element of the footplate are separate and are spaced apart from one another. Accordingly, the footplate may be referred to as a “split footplate.” The expandable element is located between the lateral element and the medial element. It secures the lateral element and the medial element of the split footplate to one another.

As its name implies, the expandable element is configured to expand. It is also configured to resiliently contract. Accordingly, the expandable element may impart the split footplate with a variety of different widths, which may enable a split footplate to accommodate feet of a plurality of different widths. Thus, a few sizes of split footplates (e.g., small, medium, large, extra large, etc.) according to this disclosure may provide a relatively custom fit for a much wider variety of foot shapes and sizes.

The lateral element and the medial element of the split footplate may comprise a somewhat rigid material. The use of a rigid material may enable one or both of the lateral element and the medial element to support a corresponding portion of the foot; i.e., a portion of the foot under which that element is configured to be positioned. In some embodiments, one or both of the lateral element and the medial element of a split footplate according to this disclosure may be configured to conform to a shape of a portion of the foot against which, or beneath which, that element is to be positioned. The degree to which an element of a split footplate conforms to a corresponding portion of a foot may vary. For example, an element of a split footplate may conform partially to the contour of the foot. Alternatively, an element of a split footplate may conform substantially to the contour of the corresponding portion of the foot; e.g., to a majority of the corresponding portion of the foot; to the general contour of the corresponding portion of the foot while standing still; to a “normal” or desired contour of the corresponding portion of the foot; etc. As another alternative, an element of a split footplate may conform completely to a contour of the foot while the foot is in a particular position and/or orientation.

The lateral element of the split footplate may be configured to prevent supination of an individual's foot and/or lateral rolling of the individual's ankle. In a specific embodiment, the lateral element of the split footplate may include a heel plate and a lateral stabilizer. The heel plate may be configured to extend from a lateral side of the foot medially to a location that will enable it to be positioned beneath (and, optionally, medially beyond) a calcaneus of the foot. The lateral stabilizer may extend upwardly from a lateral edge of the heel plate. The lateral stabilizer may be configured to extend toward or even adjacent to a lateral side of the individual's ankle.

The medial element of the split footplate may be configured to unload a medial column of the individual's foot. In this regard, the medial element may include a base that is configured to be positioned beneath the medial column. The medial element may also include an upright portion that extends upwardly from a rear portion of a medial edge of the base in manner that enables the base to be pulled upwardly.

A split footplate according to this disclosure may comprise part of a brace for supporting a foot and/or an ankle. In addition to the split footplate, such a brace may include a securing element.

In a specific embodiment, the securing element of such a brace may comprise a collar. The collar may be secured to the split footplate and is configured to hold the split footplate in place beneath an individual's foot. More specifically, the collar may be configured to be secured in place around a portion of the leg above the ankle. In addition to the split footplate and collar, a brace may include a lateral hinge and a medial hinge that secure the collar to the lateral element of the split footplate and the medial element of the split footplate, respectively. In some embodiments, one or both of the lateral hinge and the medial hinge may comprise a pivot point and a convexly oriented arcuate slot that is configured to be positioned below the malleolus of an individual's ankle.

When such an embodiment of brace is properly placed on an individual's foot and ankle, relative configurations of the collar, the lateral hinge and the lateral element of the split footplate may provide a lateral block that prevents supination of the individual's foot and/or lateral rolling of the individual's ankle. Relative configurations of the collar, the medial hinge and the medial element of the split footplate may enable the medial element of the split footplate to lift and provide support for the medial arch of the individual's foot.

In another specific embodiment, the securing element of a brace that includes a split footplate according to this disclosure may comprise a stabilizer that comprises one or more stabilizing straps, such as the stabilizers disclosed by U.S. Pat. No. 7,115,105 to Cropper (“the '105 Patent”). The stabilizer may include a foot-securing portion that is configured to be secured to the split footplate, and to secure the split footplate to an individual's foot. In addition, in embodiments where the stabilizer is configured to support or brace the individual's ankle, it may include an ankle-spanning portion that extends from the foot-securing portion to a leg-securing portion, which is configured to be secured to the individual's leg at a location above his or her ankle.

Of course, a wide variety of other embodiments of securing elements may be used with a split footplate according to this disclosure to secure it to an individual's foot and, along with the split footplate, to provide support to the individual's foot and/or ankle.

In some embodiments, a split footplate according to this disclosure and/or a brace that includes such a split footplate may be configured to be worn by an individual as he or she wears a standard shoe or other footwear. Thus, systems for supporting a foot and/or an ankle of an individual may include a split footplate according to this disclosure, an optional securing element and standard footwear.

A method for supporting a foot and/or an ankle may include selecting a split footplate having a size that generally corresponds to a shape and/or size of an individual's foot. An expandable element of a split footplate may be expanded (e.g., stretched, etc.) to increase a distance between a lateral element of the split footplate and a medial element of the split footplate, thus placing the split footplate in an open orientation. While the split footplate is in the open orientation, an individual's foot may be positioned over or on the lateral element and the medial element of the split footplate, and the expandable element may be allowed to contract, pulling the lateral element and the medial element toward one another. With the split footplate in place beneath the individual's foot, the lateral element and the medial element may be adjusted to ensure that they are properly and comfortably positioned.

With the split footplate in place relative to the individual's foot, it may be secured in place. In a specific embodiment, the split footplate may be secured in place by securing a collar to the individual's leg at a location above his or her ankle. As the collar is secured to the individual's leg, it may urge one or both of the lateral element and the medial element of the split footplate toward the other.

Other aspects, as well as features and advantages of various aspects, of the disclosed subject matter will become apparent to those of ordinary skill in the art through consideration of the ensuing description, the accompanying drawings and the appended claims.

### BRIEF DESCRIPTION OF THE DRAWINGS

In the drawings:

FIG. 1 is a front isometric view of an embodiment of an ankle brace with a split footplate, showing the ankle brace in place on a skeletal foot, ankle and lower leg;

FIG. 2 is a front view of the embodiment of ankle brace shown in FIG. 1;

FIG. 3 is a rear view of the embodiment of ankle brace shown in FIG. 1;

FIG. 4 is a top view of the embodiment of ankle brace shown in FIG. 1;

FIG. 5 is a bottom view of the embodiment of ankle brace shown in FIG. 1;

FIG. 6 is a lateral side view of the embodiment of ankle brace shown in

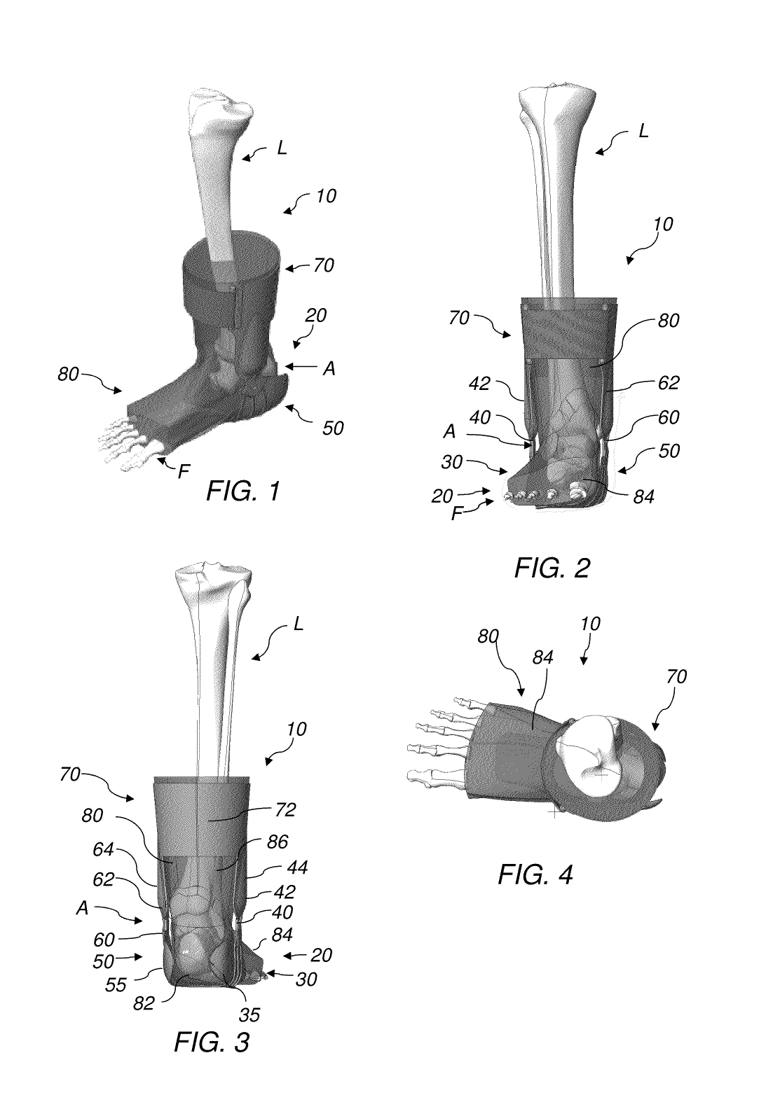

FIG. 1;

FIG. 7 is a medial side view of the embodiment of ankle brace shown in FIG. 1; and

FIG. 8 is a rear isometric view of the embodiment of ankle brace shown in

FIG. 1.

### DETAILED DESCRIPTION

FIGS. 1 through 8 provide various views of an embodiment of a brace, which is depicted as being an ankle brace 10, that is configured to be worn on an individual's ankle A, as well as on parts of the individual's foot F and lower leg L that are adjacent to the ankle A. All references in this disclosure to positioning and orientation of an ankle brace 10 (or any other embodiment of brace including a split footplate 20 according to this disclosure) and its component parts correspond to the positions and orientations when the ankle brace 10 is worn on an individual's foot F, with the sole of the foot F oriented on a horizontal surface.

Generally, the ankle brace 10 comprises a split footplate 20 with a lateral element 30 and a medial element 50, as well as an expandable element 80. The lateral element 30 is configured to be positioned on a lateral (out) side of an individual's foot F. The medial element 50 is configured to be positioned on a medial (in) side of the individual's foot F. The expandable element 80 holds the lateral element 30 and the medial element 50 of the split footplate 20 in place relative to one another, and may be configured to secure the split footplate 20 in place on the individual's foot F. The lateral element 30 and the medial element 50 of the split footplate 20 may be relatively rigid, while the expandable element 80 may comprise a material that is pliable and elastic. In embodiments where the split footplate 20 comprises part of an ankle brace 10, the ankle brace 10 may also include a securing element 70.

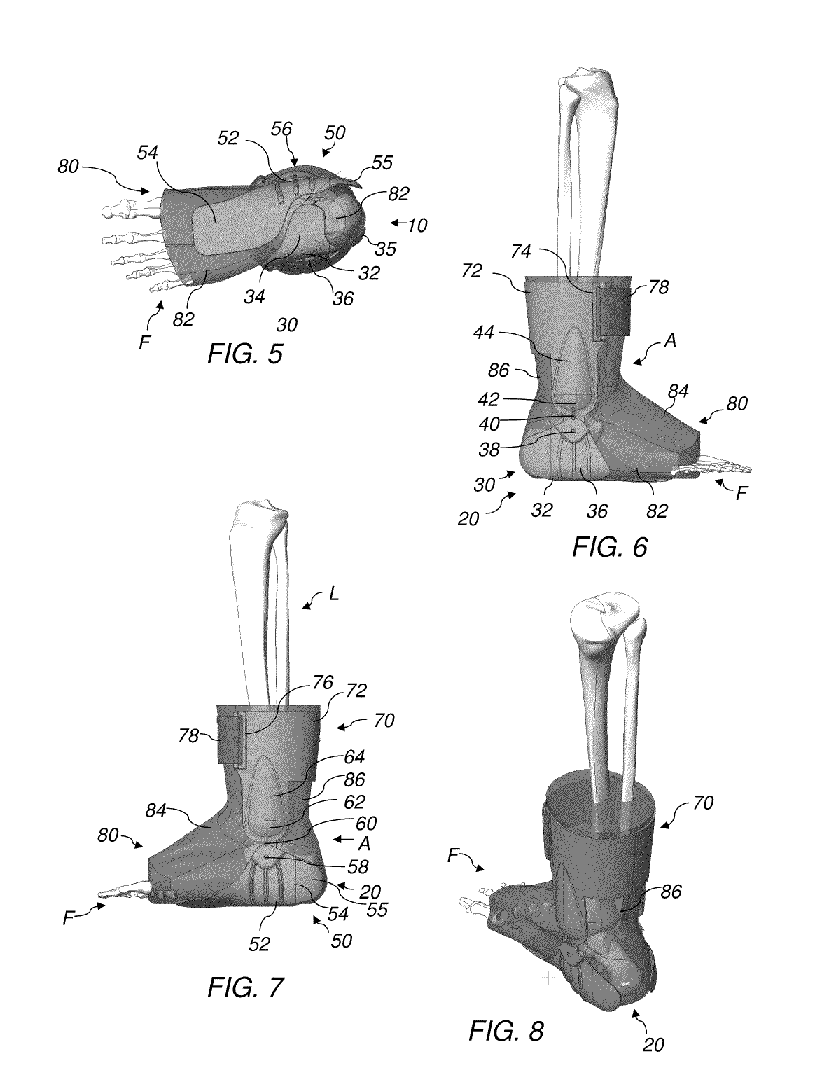

In the illustrated embodiment of split footplate 20, and as can best be seen from the views provided by FIGS. 3, 5 and 6, the lateral element 30 of the split footplate 20 includes a heel plate 34 and a lateral stabilizer 36. The heel plate 34 includes a lateral edge 32 that is configured to be aligned with and positioned beneath a lateral edge of the individual's foot F. A remainder of the heel plate 34 is configured and oriented to extend medially to a location beneath a calcaneus of the foot F. As shown in FIG. 5, the anterior extent of the heel plate 34 may be limited in such a way that the heel plate 34 may be configured not to extend beneath the transverse arch of the individual's foot F.

The lateral stabilizer 36 of the lateral element 30 of the split footplate 20 extends upwardly from the lateral edge 32 of the heel plate 34 of the lateral element 30. As depicted by FIGS. 3 and 6, the lateral element 30 may be substantially vertical oriented, and it may be configured to reside adjacent to a lateral side of the individual's ankle A.

In embodiments where the split footplate 20 is part of a hinged ankle brace 10, such as that depicted by FIGS. 1 through 8, a top portion of the lateral stabilizer 36 of the lateral element 30 may comprise a lateral hinge element 38, as illustrated by FIG. 6. The lateral hinge element 38 may define at least part of a simple pivot point or axis, or it may be part of a more complex hinge, such as the arcuate motion hinge disclosed by U.S. Patent Application Publication No. 2013/0281898 A1 of Cropper, et al., titled “FOOT AND ANKLE ORTHOSES THAT ENABLE NATURAL MOVEMENT OF THE FOOT” (“the '898 Publication”), the entire disclosure of which is hereby incorporated herein, which hinge may be configured to align with or to be substantially aligned with the tibialtalor junction, or talocrural joint, of the individuals' ankle A when the ankle brace 10 is properly positioned.

Optionally, as shown in FIG. 3, as well as by FIGS. 5 and 6, the lateral stabilizer 36 may include a posterior extension 35, which may be substantially vertically oriented, and extend posteriorly and then medially to a location around and partially behind a lateral side of the individual's heel. A posterior extension 35 with such a configuration may facilitate proper longitudinal, or anterior-posterior, positioning of the lateral element 30, and of the split footplate 20, along the length of the individual's foot F.

Referring now to FIGS. 3, 5 and 7, the medial element 50 of the split footplate 20 includes a base 54 and an upright portion 56. As illustrated by FIG. 5, the base 54 may include an anteriorly extending portion that is configured to reside beneath and to provide support to the medial column of the individual's foot F (e.g., as an upward force, etc.). The locations of the foot F beneath which the base 54 is configured to reside may differ from the locations beneath the individual's foot F beneath which the heel plate 34 of the lateral element 30 of the split footplate 20 is configured to be positioned. Thus, the base 54 and the heel plate 34 may lack any overlapping, or superimposed, regions. The base 54 and the heel plate 34 may even be laterally spaced apart from one another. In the illustrated embodiment, at posterior location, the base 54 of the medial element 50 accommodates the heel plate 34 of the lateral element 30 of the split footplate 20; thus, the posterior portion of the base 54 resides primarily beneath a medial edge of the sole of the individual's foot F.

The upright portion 56 of the medial element 50 of the split footplate 20 extends upwardly from a medial edge 52 of the base 54 of the medial element 50. More specifically, the upright portion 56 extends upwardly from a portion of the medial edge 52 that is configured to be positioned below a medial side of the ankle A. Thus, the upright portion 56, which may be oriented substantially vertically, may be configured to extend over the medial side of the ankle A.

In embodiments where the split footplate 20 is part of a hinged ankle brace 10, such as that depicted by FIGS. 1 through 8, a top portion of the upright portion 56 of the medial element 50 may comprise a medial hinge element 58, as illustrated by FIG. 7. The medial hinge element 58 may be configured to at least partially define a simple pivot point or axis, or it may be part of a more complex hinge, such as the arcuate motion hinge disclosed by the '898 Publication.

The upright portion 56 of the medial element 50 may optionally include a posterior extension 55, as depicted by FIG. 3, and by FIGS. 5 and 7. The posterior extension 55 of the upright portion 56 may be substantially vertically oriented, and may extend posteriorly and then laterally to a location around and partially behind a medial side of the individual's heel. A posterior extension 55 with such a configuration may facilitate proper longitudinal, or anterior-posterior, positioning of the medial element 50, and of the split footplate 20, along the length of the individual's foot F.

The lateral element 30 and the medial element 50 of the split footplate 20 may comprise a somewhat rigid material or combination of materials. It addition, each of the lateral element 30 and the medial element 50 may be formed from a material that provides a desired degree of one or more other characteristics, such as flexibility, strength, durability and/or fracture mechanics. Examples of suitable materials include, but are not limited to, thermoplastic materials (e.g., polypropylene, polyethylene, etc.), thermoplastic composite materials (e.g., carbon fiber-reinforced thermoplastic materials, etc.) and resins (e.g., a polypropylene-like material, such as the DurusWhite™ material available from Objet Geometries, Ltd., of Rehovot, Israel, etc.). In some embodiments, features that are formed from a relatively hard material may be coated with a softer, more compliant material (e.g., natural rubber, silicone, a compliant thermoplastic material (or oil-extended thermoplastic material), a cushioning material (e.g., ethylene-vinyl acetate (EVA) foam, or foam rubber; etc.), etc.).

The expandable element 80 of the split footplate 20 may hold the lateral element 30 and the medial element 50 in place relative to one another. In some embodiments, the expandable element 80 may be located laterally between adjacent sections of the split footplate 20 (e.g., the lateral element 30, the medial element 50, etc.). In other embodiments, the expandable element 80 may be configured as a sock or a stocking on which or in which other elements of the split footplate 20 reside.

In addition, the expandable element 80 is configured to impart the split footplate 20 with a variety of different widths (e.g., distances between the lateral element 30 and the medial element 50, etc.), which may enable a split footplate 20 to accommodate feet of a plurality of different widths. In some embodiments, the expandable element 80 may comprise a fabric or another material that can expand and resiliently contract. Breathability may also be a desirable characteristic of the expandable element 80. A variety of materials may be used to form the expandable element 80, including, without limitation, stretchable, elastic materials, such as the laminate material available from Cropper Medical, Inc., of Ashland, Oreg., under the BIOSKIN® trademark, which material includes a polyurethane membrane sandwiched between two layers of a four-way stretchable fabric, such as spandex, or elastane, or a stretchable, elastic nylon.

As shown in FIG. 5, a base section 82 of the expandable element 80 is configured to be positioned beneath the individual's foot F. At least part of the base section 82 is located between opposed, or facing, edges of the heel plate 34 of the lateral element 30 and the base 54 of the medial element 50 of the split footplate 20.

FIGS. 4 and 6 illustrate an upper section 84 of the expandable element 80, which may extend between an upper anterior edge of the lateral stabilizer 36 of the lateral element 30 of the split footplate 20 and an upper anterior edge of the upright portion 56 of the medial element 50 of the split footplate 20. As shown, the upper section 84 of the expandable element 80 may be configured to reside atop the individual's foot F.

In addition, the expandable element 80 may include a posterior portion 86, as depicted by FIGS. 3, 6, 7 and 8. The posterior portion 86 may extend between upper and posterior edges of the lateral stabilizer 36 of the lateral element 30 and of the upright portion 56 of the medial element 50. Thus, the posterior portion 86 of the expandable element 80 may be configured to be positioned around the back of an individual's heel and/or ankle A.

In addition to the split footplate 20, an ankle brace 10 or other embodiments of brace that includes a split footplate 20 may also include a securing element 70, which may secure the brace in place relative to a particular portion of an individual's anatomy (e.g., his or her ankle, etc.). The embodiment of ankle brace 10 shown in FIGS. 1 through 8 includes a specific, but non-limiting, example of a securing element 70 (see FIGS. 1-4, 7 and 8) that may be configured for use with a split footplate 20 according to this disclosure.

The securing element 70 is configured to be secured around an individual's lower leg L. The securing element 70 may include a collar 72 that is configured to extend around the posterior side, or back, of an individual's lower leg L (e.g., his or her calf, etc.). Sides of the collar 72 may extend anteriorly and, thus, be configured to be positioned over sides of the lower leg L. At its anterior edges, or front edges, the collar 72 may include strap engagement elements 74 (FIGS. 6) and 76 (FIG. 7). The strap engagement elements 74 and 76 may be configured to receive or otherwise engage portions of an elongated element, such as a strap 78 (e.g., a strap with a touch fastener, etc.), which may be configured to pull the collar 72 tightly against the back and sides of the lower leg L and, thus, to fasten the securing element 70 in place around the lower leg L.

As best seen in FIGS. 3, 6 and 7, the depicted embodiment of securing element 70 includes a lateral hinge element 42 (FIGS. 3 and 6) and a medial hinge element 62 (FIGS. 3 and 7) that extend downwardly from respective lateral and medial sides of the collar 72. In some embodiments, such as the illustrated embodiment, the lateral hinge element 42 and the medial hinge element 62 may be located at the bottom ends of a lateral support member 44 and a medial support member 64 of the collar, which respectively extend downwardly from lateral and medial sides of the collar 72.

Each of the lateral hinge element 42 and the medial hinge element 62 may be configured to cooperate with its corresponding lateral hinge element 38 or medial hinge element 58 of the split footplate 20. Together (i.e., when assembled), the lateral hinge elements 38 and 42 may define a lateral hinge 40 (FIGS. 3 and 6), while the medial hinge elements 58 and 62 may define a medial hinge 60 (FIGS. 3 and 7). Thus, the lateral hinge 40 and the medial hinge 60 couple the securing element 70 to the split footplate 20.

The collar 72, the lateral support member 44 and the medial support member 64 of the securing element 70 may be substantially rigid, and may be constructed from any suitable material, such as a thermoplastic material, a thermoplastic composite material, a resin or any other suitable material. In addition, surfaces of one or more of these features that are configured to be positioned against part of an individual's lower leg L may be lined with a material that provides cushioning, a conformal fit or any other desired function.

An embodiment of ankle brace such as that depicted by FIGS. 1 through 8 may be configured to support the individual's ankle A. More specifically, when the split footplate 20 is worn on the individual's foot F and the securing element 70 is secured to the individual's lower leg L, the components and the materials from which they are made may provide a combination of a customized fit (even though the ankle brace 10 may not be custom-made), support, freedom of motion and comfort. Without limitation, the rigid material and configurations of the lateral element 30 and the medial element 50 may support the foot F, while conforming to the shapes of the portions of the foot F against which, or beneath which, the lateral element 30 and the medial element 50 are positioned. Even more specifically, when the heel plate 34 is forced against an individual's heel, the heel plate 34 and the lateral stabilizer 36 of the lateral element 30 may, alone or in combination with the lateral support member 44 of the securing element 70, be configured to prevent supination of the individual's foot F and/or lateral rolling of the individual's ankle A.

In addition, when the ankle brace 10 is positioned on an individuals' foot F, over his or her ankle A and around his or her lower leg L, the heel plate 34 of the lateral element 30 provides an anchor that defines a position along the lower leg L. When the securing element 70 is snugly and properly fastened to the lower leg L, it pulls the medial element 50 of the split footplate 20 upward, generating a tension in the base 54 of the medial element 50. That tension causes the base 54 to pull the medial column of the foot F upward, or unloads the medial column.

Another specific embodiment of a brace according to this disclosure may include a split footplate, such as the embodiment of split footplate 20 depicted by FIGS. 1 through 8, as well as a stabilizer with one or more stabilizing straps, such as those disclosed by the '105 Patent. The stabilizer may include a foot-securing portion that is configured to be secured to the split footplate, and to secure the split footplate to an individual's foot. In addition, in embodiments where the stabilizer is configured to support or brace the individual's ankle, it may include an ankle-spanning portion that extends from the foot-securing portion to a leg-securing portion, which is configured to be secured to the individual's leg at a location above his or her ankle. Such a stabilizer, when used in combination with a split footplate 20, may enable independent unloading of the medial and lateral columns of the foot F, as well as controlled unloading of each of the medial column and the lateral column.

Although the preceding disclosure provides many specifics, these should not be construed as limiting the scope of any of the ensuing claims. Other embodiments may be devised which do not depart from the scopes of the claims. Features from different embodiments may be employed in combination. The scope of each claim is, therefore, indicated and limited only by its plain language and the full scope of available legal equivalents to its elements.

What is claimed:

1. A brace for use with a foot and/or ankle of an individual, comprising:
a footplate including:
a lateral element;
a medial element separate from and spaced apart from the lateral element; and
an expandable element between the lateral element and the medial element, the expandable element configured to enable the footplate to accommodate feet of a plurality of different widths.

2. The brace of claim 1, wherein the lateral element of the footplate and the medial element of the footplate are somewhat rigid.

3. The brace of claim 1, wherein the expandable element comprises an elastic, resilient material extending between the lateral element and the medial element.

4. The brace of claim 3, wherein the expandable element is configured to at least partially conform to a shape of a bottom of the foot of the individual.

5. The brace of claim 1, further comprising:
a collar configured to be secured to a leg of an individual at a location above an ankle; and
at least one hinge securing the footplate to the collar.

6. The brace of claim 5, wherein the at least one hinge is configured for adjustment between a plurality of pivot points.

7. The brace of claim 5, wherein the lateral element of the footplate includes:
a heel plate that extends medially to a position configured to be located beneath a calcaneus of a foot of an individual; and
a lateral stabilizer extending upward from a lateral edge of the heel plate.

8. The brace of claim 7, wherein the at least one hinge comprises a lateral hinge that couples the lateral stabilizer of the lateral element of the footplate to a lateral side of the collar.

9. The brace of claim 8, wherein the heel plate, the lateral stabilizer and the lateral hinge are collectively configured to block supination and/or lateral rolling of an ankle as a calcaneus of a foot is positioned on the heel plate and the ankle is positioned adjacent to the lateral hinge.

10. The brace of claim 9, wherein the collar, when secured to the leg of the individual, enhances blocking of supination and/or lateral rolling of the ankle as the calcaneus of the foot is positioned on the heel plate and the ankle is positioned adjacent to the lateral hinge.

11. The brace of claim 9, wherein the heel plate, the lateral stabilizer and the lateral hinge enable movement of the foot about the ankle in the sagittal plane.

12. The brace of claim 5, wherein tension in the medial element, the at least one hinge and the collar are collectively configured to unload a medial column of a foot of an individual.

13. The brace of claim 12, wherein the medial element includes:
a base configured to be positioned beneath the medial column; and
an upright portion configured to be secured to the at least one hinge, the at least one hinge comprising a medial hinge.

14. The brace of claim 13, wherein the base of the medial element includes:
an arch support that extends laterally and anteriorly, and, when under tension, is configured to support a medial arch of the foot of the individual and unload the medial column of the foot.

15. The brace of claim 1, further comprising:
a stabilizing strap including:
a foot-securing portion configured to be secured to the footplate;
an ankle-spanning portion configured to extend substantially linearly over the ankle; and
a leg-securing portion configured to be secured to a portion of a leg above the ankle.

16. The brace of claim 15, wherein the foot-securing portion is configured to be secured to a bottom of the footplate.

17. The brace of claim 15, wherein the foot-securing portion is configured to wrap around the footplate.

18. The brace of claim 1, further comprising:
a collar configured to be secured to a leg, above the ankle;
a lateral hinge pivotally connecting a lateral side of the upper cuff to the lateral element; and
a medial hinge pivotally connecting a medial side of the upper cuff to the medial element.

19. A brace for use with a foot and/or ankle, comprising:
a footplate including:
a lateral element;
a medial element separate from and spaced apart from the lateral element; and
an expandable element between the lateral element and the medial element, the expandable element configured to enable the footplate to accommodate feet of a plurality of different widths;
an upper cuff;
a lateral hinge pivotally connecting a lateral side of the upper cuff to the lateral element; and
a medial hinge pivotally connecting a medial side of the upper cuff to the medial element, the medial hinge configured for adjustment between a plurality of pivot points.

20. The brace of claim 19, wherein:
the lateral element includes:
a block that extends medially and is configured to be located beneath a calcaneus of a foot of an individual; and
a collar extending upwardly from a lateral portion of the block; and
the medial element includes:
a base configured to be located beneath a portion of the foot of the individual, the base including:
an arch support that extends laterally and anteriorly, and is configured to support a medial arch of the foot of the individual; and
a collar extending upwardly from a medial portion of the base.

21. A method for supporting a foot and/or an ankle, comprising:
selecting a footplate having a foot bed with an interior width that is less than a width of a foot with which the footplate is to be used;
positioning a foot on the footplate, with an arch of the foot being placed over and supported by a medial element of the footplate and a heel of the foot being placed over a lateral element of the footplate, the foot urging the lateral element and the medial element apart from one another, while an expandable element between the lateral element and the medial element enables the urging while resiliently urging the lateral element and the medial element toward one another.

22. The method of claim 21, further comprising:
securing a collar secured to the lateral element and the medial element in place around a leg at a location above an ankle adjacent to the foot.

23. The method of claim 22, wherein the act of securing the collar further urges the lateral element and the medial element toward one another.

24. The method of claim 22, wherein, upon securing the collar, the collar, a hinge located adjacent to a lateral side of the ankle and the lateral element collectively prevent rolling of the ankle while allowing for movement of the foot about the ankle in a sagittal plane.

25. The method of claim 22, wherein, upon securing the collar, the collar, a hinge located adjacent to a medial side of the ankle and the medial element collectively unload a medial column of the foot.

26. The method of claim 21, further comprising:
positioning the foot and the footplate within footwear.

27. A system for supporting a foot and/or an ankle, comprising:
standard footwear having a size configured to normally fit a foot of an individual; and
a brace configured, when worn on the foot, to fit within the standard footwear, the brace including:
a footplate including:
a lateral element;
a medial element separate from and spaced apart from the lateral element; and
an expandable element between the lateral element and the medial element, the expandable element configured to enable the footplate to accommodate feet of a plurality of different widths.

28. The system of claim 27, wherein the brace further includes:
a collar associated with the footplate and configured to be secured to a leg of the individual, on an opposite side of an ankle associated with the foot.

29. The system to claim 28, wherein the brace further includes:
at least one hinge for coupling the collar to the footplate.

30. The system of claim 28, wherein the collar comprises a portion of a strap that is configured to be secured in place relative to the footplate.

31. The system of claim 28, wherein the collar and the lateral element of the footplate are configured to collectively block supination and/or lateral rolling of the ankle.

32. The system of claim 28, wherein the collar and the medial element of the footplate are configured to collectively unload a medial column of the foot.
